(19) United States
(12) Patent Application Publication
(10) Pub. No.: US 2014/0072617 A1
(43) Pub. Date: Mar. 13, 2014

(54) METHOD FOR PREVENTING OR SLOWING DOWN THE APPEARANCE OF THE EFFECTS OF SKIN AGEING USING A TOCOPHERYL PHOSPHATE IN LIPOSOMES

(71) Applicant: LVMH RECHERCHE, Saint Jean de Braye (FR)

(72) Inventors: Marc DUMAS, Saint Jean le Blanc (FR); Emmanuelle NOBLESSE, Donnery (FR); Valérie ALARD, Orleans (FR); Dominique QUILES, Chenault (FR); Eric PERRIER, Les Cotes D'Arey (FR)

(73) Assignee: LVMH RECHERCHE, Saint Jean de Braye (FR)

(21) Appl. No.: 14/046,481

(22) Filed: Oct. 4, 2013

Related U.S. Application Data
(63) Continuation-in-part of application No. 12/284,851, filed on Sep. 25, 2008.

(30) Foreign Application Priority Data
Sep. 26, 2007 (FR) 0706750

(51) Int. Cl. A61K 8/55; A61Q 19/08

(52) U.S. Cl. CPC A61K 8/55; A61Q 19/08
USPC 424/450; 514/100

(57) ABSTRACT

The invention relates to a method for preventing or slowing down the appearance of the effects of skin ageing, in particular for slowing down the formation of wrinkles on the face, and reducing or smoothing out the wrinkles already formed, using a tocopheryl phosphate in liposomes.

This application is a Continuation In Part to pending U.S. patent application Ser. No. 12/284,851, filed on Sep. 25, 2008.

The invention relates to a novel use of tocopheryl phosphate, in particular in its dl or d form, or of an ester thereof or of a salt thereof, which is cosmetically or pharmaceutically acceptable, in the cosmetics field, and also to the compositions containing same and to the cosmetic methods using them.

Each day, the skin is subjected to considerable mechanical stresses.

Observations have shown that skin ageing leads to a loss of cell density in the superficial layers of the skin.

The loss of tissue material (collagen and other dermal matrix proteins) brought about by this absence of cells is reflected by less tissue cohesion and also by a thinner epidermis with fewer cell strata.

The stress to which the skin is subjected, in particular UV exposure, leads to a deterioration and then a natural elimination of numerous epidermal and dermal cells.

The many histological observations made in order to understand the key phenomena of dermal ageing have in particular made it possible to conclude that there is a loss of dermal cell density in the wrinkled light-exposed areas.

This loss largely explains the loss of tissue material (collagens and other proteins of the dermal matrix and of the dermal-epidermal junction) that is observed during ageing, since this material can no longer be produced and deposited under the wrinkle by absent cells.

At the epidermal layer, under the wrinkles, this depletion of cells is also observed (Contet-Audonneau et al., A histological study of human wrinkle structures: comparison between sun-exposed areas of the face, with or without wrinkles, and sun-protected areas. Br J. Dermatol. 1999 June; 140(6):1038-47) and is reflected, at the histological level, by a thinner epidermis with fewer cell strata in the hollow of the wrinkles.

This phenomenon involves most particularly the cells of the epidermal basal layer.

In fact, although the considerable undulations of the dermal-epidermal junction (DEJ) in young skin support a large basal cell population, these undulations of the DEJ disappear with age, this interface then supporting only a small basal cell population.

Furthermore, preliminary observations imply that, in wrinkled light-exposed areas, a similar phenomenon of decrease in the basal cell population occurs.

This phenomenon, which affects in particular the cells of the basal layers of the epidermis, is important because it is these cells which ensure the formation and the renewal of the epidermal cells, and are themselves derived from the division of “epidermal stem cells”.

These epidermal stem cells are present in the basal layers of the epidermis and are defined as being cells with a high regenerative potential, which have the particularity of being able to give an unlimited descendance of “daughter” cells.

Maintaining these epidermal stem cells in the basal layers of the epidermis is therefore a major target for preventing or slowing down the loss of cell density in the epidermis as observed during photoageing of the skin.

The Applicant has now shown that it is possible to protect the stem cells of the epidermis by means of a treatment using tocopheryl phosphate, and in particular alpha-tocopheryl phosphate, especially in its dl or d form, or an ester thereof or a salt thereof, which is cosmetically or pharmaceutically acceptable.

Tocopheryl phosphate is a molecule which was described by the Applicant, in patent application WO 91/11189 which matured notably as U.S. Pat. No. 5,387,579, for its use in the preparation of a pharmaceutical or cosmetic or dermatological composition for the prevention or treatment of allergic manifestations such as skin allergy or bronchial or inflammatory asthma, or else for the prevention or treatment of the harmful effects of free radicals.

By virtue of the studies by the inventors of the present application, it has been shown that tocopheryl phosphate, in particular alpha-tocopheryl phosphate, can be used as an active agent in cosmetic compositions which aim at preserving the basal cells of the epidermis and the epidermal stem cells thereof, which are the only cells capable of dividing and of giving numerous generations of “daughter” cells, and thus of having a positive impact on epidermal regeneration, reflected by the maintaining of a sufficient cell density in the various layers of the epidermis and of the protein constituents that these cells produce.

The present invention thus relates, according to a first aspect, to the use of tocopheryl phosphate, and in particular of alpha-tocopheryl phosphate, especially in its dl or d form, or of an ester thereof or of a salt thereof, which is cosmetically or pharmaceutically acceptable, as an active agent for preventing or slowing down skin ageing in particular related to the effects of UV exposure or photoageing, in cosmetic compositions.

In the various aspects of the invention, the tocopheryl phosphate will especially be in its dl form or in its d form or in the form of a cosmetically acceptable salt or ester thereof.

According to a particularly advantageous variant, the tocopheryl phosphate is alpha-tocopheryl phosphate.

The tocopheryl phosphate, in particular the alpha-tocopheryl phosphate, may be in the form of cosmetically acceptable salts chosen from the group consisting of alkali metal salts, in particular monosodium or disodium salts, alkaline earth metal salts, in particular magnesium salts, and ammonium salts or salts of primary, secondary or tertiary amines such as, in particular, diethylamine, diethanolamine, triethylamine or triethanolamine.

According to a second aspect, the invention also relates to a cosmetic composition containing a tocopheryl phosphate as defined above.

According to a third aspect, the invention relates to a cosmetic skin care method for preventing or slowing down the appearance of the effects of skin ageing by applying, to the areas of the skin in need thereof, a composition comprising, as one of the active agents, a tocopheryl phosphate, in particular an alpha-tocopheryl phosphate.

For any aspect, according to a particular feature, said tocopheryl phosphate is at least partially encapsulated in liposomes.

The liposomes may be unilamellar or multilamellar. The liposomes used according to the invention have a diameter of approximately 150 to 250, in particular 150 to 200, Nanometers (nm), as measured by laser granulometry as a suspension of lipid vesicles or by Transmission Electron Microscopy with preparation of the sample by cryofracture.

According to another particular feature, the invention relates to a cosmetic composition consisting of or comprising a continuous aqueous phase in which liposomes are present, containing a tocopheryl phosphate, in particular in the form of a cosmetically acceptable salt or ester thereof as defined above, said tocopheryl phosphate being at least partially incorporated into these liposomes, and said composition also comprising at least one water-soluble polysaccharide.

According to a variant, the invention cosmetic compositions contain a tocopheryl phosphate, in particular an alpha-tocopheryl phosphate, at least partially encapsulated in liposomes which are themselves contained in an aqueous phase.

According to a first variant, the cosmetic composition consists essentially of an aqueous phase in which the liposomes are present.

According to a second variant, the cosmetic composition contains a continuous aqueous phase in which the liposomes are present and which is more specifically in the form of an oil-in-water emulsion.

Thus, according to a variant, the compositions are in the form of an oil-in-water emulsion proved to be particularly advantageous in the cosmetic skin cares targeted. The fatty phase of the emulsion may advantageously contain at least one triglyceride.

According to a further variant of the invention, the fatty phase may itself be in the form of a water-in-oil (W/O) emulsion. In such a case the composition is in the form of at lesat a double emulsion well known to one skill in the art.

According to a further variant of the composition, the liposomes are stabilised by the presence of an alginate in the aqueous phase.

According to another particular feature of the invention, the liposomes are comprising a phospholipid membrane comprising a mixture of two different fractions of soybean lecithin, a first fraction of soybean lecithin being a mixture of phospholipids comprising more than 90 weight % of phosphatidyl choline, and a second fraction of soybean lecithin being a mixture of phospholipids comprising between 15% and 30%, by weight of phosphatidylcholine.

According to a particular embodiment, the composition of the second fraction of soybean lecithin comprises:

From 10 to 20% of Phosphatidyl ethanolamine

From 10 to 15% of Phosphatidyl inositol From 15 to 30% of Phosphatidyl choline

From 5 to 10% of phosphatidic acid

Less than 1% of Lyso Phosphatidyl choline.

According to another particular embodiment, the composition of the second fraction comprises, in weight %:

From 13 to 18% of Phosphatidyl ethanolamine

From 12 to 15% of Phosphatidyl inositol

From 22 to 30% of Phosphatidyl choline

From 5 to 10% of phosphatidic acid

Less than 1% of Lyso Phosphatidyl choline.

The first fraction of soybean lecithin comprises typically the following composition in weight %: From 90 to 95% of Phosphatidyl choline, and is available on the market under the Trade Mark of Emulmetik® 930, sold by the Company Lucas Meyer,

The second fraction of soybean lecithin, which is available on the market underr the Trade mark Emulmetik® 300, also sold by Lucas Meyer, typically comprises the following composition in weight %:

From 13 to 18% of Phosphatidyl ethanolamine

From 12 to 15% of Phosphatidyl inositol

From 22 to 30% of Phosphatidyl choline

From 5 to 10% of phosphatidic acid

Less than 1% of Lyso Phosphatidyl choline,

According to the invention other lipids can be used to form the liposomes consisting of further amphiphilic lipids, i.e. molecules having a hydrophilic group which is indifferently ionic or non-ionic, and a lipophilic group.

In the present description and the claims, the term “lipid” covers all substances comprising a lipophilic group, the “fatty” carbonaceous chain of which contains more than 5 carbon atoms.

Similarly, for the purpose of the invention, the term “fatty alcohols” is intended to mean alcohols of which the carbonaceous chain contains at least 5 carbon atoms.

The amphiphilic lipids may be phospholipids, phosphoaminolipids, glycolipids, or mixtures of these lipids. Such substances consist, for example, of an egg or soybean lecithin, a sphingomyelin, a cerebroside or an oxyethylenated polyglyceryl stearate.

The phospholipids may be phosphatidylcholine, phosphatidylserine, phosphatidylethanolamine, phosphatidylglycerol, phosphatidylinositol, or mixtures thereof.

The composition may also comprise a non-ionic surfactant advantageously consisting of a mixture of fatty alcohol polyalkylene glycol ethers advantageously having different HLBs.

The non-ionic surfactant may be chosen from fatty alcohol polyethylene glycol ethers and mixtures thereof, in particular ethoxylated derivatives of the stearyl alcohol of formula (A) and mixtures thereof.

CH₃(CH₂)₁₇(OCH₂CH₂)ₙOH (A)

In a particularly advantageous embodiment, a mixture of non-ionic surfactants, one being rather of substantially hydrophilic nature and the second being rather of substantially lipophilic nature, is used.

In a particular embodiment of the invention, the non-ionic surfactant is a mixture of non-ionic surfactants comprising steareth-2, in accordance with formula (A) and in which n=2 on average, sold under the name Brij® 72 and the HLB value of which is 4.9, and steareth-21, in accordance with formula (A) and in which n=21 on average, sold under the name Brij® 721P and the HLB value of which is 15.3.

The steareth-2/steareth-21 ratio is adjusted so as to stabilize the emulsion without degrading the liposomes.

According to another particular feature, the aqueous phase containing the liposomes in dispersion, or the aqueous phase of an oil-in-water emulsion, may contain at least one first water soluble polysaccharide, which was recognized as markedly increasing the stability of the liposomes.

The polysaccharide(s) in water-soluble form may be chosen from a wide range of water-soluble polysaccharides.

The polysaccharide concentration will be chosen so as to effectively protect the liposomes against degradation thereof.

They may in particular be chosen from the group consisting of starch or a derivative thereof, cellulose derivatives, pectins, gums, alginate, dextrans, carragheenates and hyaluronic acid.

Among the cellulose derivatives, carboxymethylcellulose, hydroxymethylcellulose, cellulose acetate or methylcellulose will in particular be chosen.

Among the gums, xanthan gum or guar gum will in particular be chosen.

However, alginates, in particular alkali metal alginates, and most particularly a sodium or potassium salt or an extract containing same, for example an algal extract, will advantageously be chosen.

In a particular embodiment, the composition contains at least one alkali metal alginate and at least a second polysaccharide in water-soluble form, in particular an alkali metal salt of carboxymethylcellulose, preferably sodium carboxymethylcellulose.

The polysaccharide concentration will be chosen so as to effectively protect the liposomes against their degradation under the effect of the non-ionic surfactants present in the continuous aqueous phase and intended to stabilize the emulsion.

In one embodiment, the total amount of water-soluble polysaccharide is ranging between 0.1% and 10% by weight of the composition, better between 0.1% and 2% by weight.

In another embodiment care will also be taken to ensure that the [phospholipids/water-soluble polysaccharide] ratio of the composition is between 0.1 and 20, in particular between 1 and 10.

The cosmetic composition according to the invention which comprises at least one first water-soluble polysaccharide may also advantageously contain at least one hydrophilic polymer other than the polysaccharide, which can be chosen from the group consisting of polyvinylpyrrolidone and polyvinyl alcohol, and any mixtures thereof.

In addition, the cosmetic composition according to the invention may comprise other water-soluble hydrophilic compounds.

The soluble hydrophilic compounds may, for example, be a C₆ or C₁₂ sugar, advantageously chosen from glucose, sorbitol, sucrose, lactitol, glycerol or one of their ethers or esters or of their derivatives.

These water-soluble compounds are preferably obtained from a plant extract.

In one variant of the invention, the cosmetic composition may comprise one or more cosmetically active agents.

Those skilled in the art will understand that the choice of this (or these) cosmetically active agent(s) depends of course on the desired cosmetic properties.

They will also easily understand that, depending on the nature of this agent, it will be present either in the fatty phase or in the aqueous phase, or at least partially incorporated into the liposomes.

Preferably, at least a part of the cosmetically active agents is encapsulated in the liposomes present in the cosmetic composition.

The cosmetically acceptable active agent then becomes concentrated in the hydrophilic or hydrophobic compartments of the liposome according to its own affinity.

Active agents having a strong affinity for lipids are thus preferentially located in the hydrophobic intramembrane spaces, whereas hydrophilic active agents, for example in ionic form, are located in the hydrophilic intermembrane spaces, and in the particular case of liposomal vesicles, also in their core.

The cosmetically active agent encapsulated in the liposomes may advantageously be chosen from the group consisting of substances having a skin-depigmenting activity or a skin-lightening activity; substances having a slimming activity; substances having a hydrating activity; substances having a calming, soothing or relaxing activity; substances having an activity stimulating the microcirculation of the skin, so as to improve the radiance of the complexion, in particular of the face; substances having a sebum-regulating activity for oily skincare; substances for cleansing or purifying the skin; substances having a free-radical scavenger activity or substances having an anti-ageing activity.

The composition according to the invention may advantageously comprise several cosmetically active substances chosen from the same group or else chosen from groups of substances having a different cosmetic effect.

As substance with skin-depigmenting or skin-lightening activity, use is preferably made of a substance chosen from the group consisting of ascorbic acid derivatives, especially esters such as ascorbyl glucosides and ascorbyl phosphates, in particular magnesium ascorbyl phosphate, and an extract of black elder (Sambucus nigra) fruits or flowers.

The substance with skin-depigmenting or skin-lightening activity will advantageously be used at a concentration of between 0.001% and 5%, and in particular between 0.01% and 3% by weight, relative to the total weight of the composition.

As substance with a hydrating effect, use may, for example, be made of glycerol, at least one alcohol such as an alkylene glycol, and in particular propylene glycol, butylene glycol, pentylene glycol and mixtures thereof in any proportions, in particular those commercially available of PEG-60 type. The composition will advantageously contain from 0.001% to 5% by weight, preferably from 0.1% to 5%.

As substance with a calming, soothing or relaxing effect, use may, for example, be made of a glycyrrhizate, in particular in the form of a potassium salt.

As substance with free-radical scavenger or anti-ageing activity, use may, for example, be made of a tocopheryl acetate in addition to said tocopheryl phosphate.

According to a particular variant embodiment, the proportion by weight of each of these substances will be between 0.001% and 5%.

As substance for reducing wrinkles, use may advantageously be made of the palmitoyl pentapeptide-3 sold under the name (Remixyl®) Matrixyl, in particular (Remixyl) Matrixyl® 3000, an extract of mallow (Vitactyl®), an extract of maize grain (Deliner®, Zea mays kernel extract), or an extract of oat bran (Osilift®, Avena sativa bran extract).

As substance with a slimming effect, use may, for example, be made of xanthine, such as caffeine. According to a particular variant embodiment, the proportion by weight of substance with a slimming effect will be between 0.001% and 5%.

As substance stimulating the microcirculation of the skin, use may, for example, be made of ruscogenin.

According to a particular variant embodiment, the proportion by weight of substance stimulating the microcirculation of the skin will be between 0.001% and 5%.

As substance with sebum-regulating activity for oily skincare, use may, for example, be made of zinc oxide or at least one zinc-based derivative, in particular the organic zinc salts, such as zinc gluconate, zinc salicylate or zinc pidolate.

According to a particular variant embodiment, the proportion by weight of substance with sebum-regulating activity will be between 0.01% and 10%.

According to yet another particular embodiment of the invention, it may be advantageous to add, to said cosmetic composition, at least one refreshing agent such as, for example, menthol or a derivative thereof, such as menthoxypropanediol.

In another embodiment of the invention, the oily phase of said emulsion may comprise triglycerides.

According to a particularly advantageous variant of the invention, the compositions of the invention may be in the form of a multiple emulsion in which the fatty phase of the emulsion is itself a water-in-oil (W/O) emulsion.

Finally, the composition according to the invention may also comprise, in addition, one or more cosmetically acceptable excipients chosen from the group consisting of pigments, dyes, rheological agents, fragrances, sequestering agent, electrolytes, pH adjusters, antioxidants, preservatives, and mixtures thereof, texturing agents, and antisun agents or sunscreens.

The present invention also relates to a method for preparing the cosmetic composition defined above, which method comprises the following steps:
preparing an aqueous phase containing said liposomes and at least one polysaccharide in water-soluble form;
preparing a stock emulsion stabilized with said non-ionic surfactant, and
mixing said stock emulsion with said aqueous phase containing the liposomes, thereby obtaining a stable emulsion.

In a preferred embodiment of the method according to the invention, the polysaccharide is brought into contact with a hydrated lipid phase prior to the step of forming the liposomes by shearing.

Thus, according to a first particularly advantageous variant of this method, it comprises the following successive steps:
preparing a hydrated lamellar lipid phase,
incorporating said hydrated lamellar lipid phase into an aqueous solution comprising at least one water-soluble polysaccharide,
forming the liposomes by shearing the mixture above and
incorporating the mixture above into the stock emulsion containing said non-ionic surfactant.

In the above variant of the method, the preparation of a hydrated lamellar phase is obtained by preparing a concentrated surfactant phase in which the amphiphilic lipids are dissolved in glycerol and 1,3-butylene glycol and then water so as to form a hydrated lamellar phase.

Next, the hydrated lamellar phase previously formed is incorporated into an aqueous solution comprising the polysaccharide(s), vigorously stirred, for example with an Ultra Turrax, so as to form an aqueous dispersion of liposomes.

According to this variant, a fraction of the total water-soluble polysaccharide amount can be incorporated directly into the stock solution.

By applying this first variant of the preparation method, a fraction of the amount of polysaccharides can be encapsulated into said liposomes, the other fraction then being dissolved in the free state, i.e. “non-encapsulated”, in the aqueous phase.

According to a second particularly advantageous variant of this method, it comprises the following successive steps:
preparing a hydrated lamellar lipid phase,
forming the liposomes by shearing the mixture above,
incorporating the dispersion of liposomes into an aqueous solution comprising at least one water-soluble polysaccharide, and
incorporating the mixture above into the stock emulsion containing said non-ionic surfactant.

In the above variant of the method, the preparation of a hydrated lamellar phase is obtained by preparing a concentrated surfactant phase in which the amphiphilic lipids are dissolved in glycerol and 1,3-butylene glycol and then water, so as to form a hydrated lamellar phase.

Next, the hydrated lamellar phase previously formed is vigorously stirred, for example with an Ultra Turrax, so as to form an aqueous dispersion of liposomes.

The dispersion of liposomes is then incorporated into an aqueous solution comprising the alkali metal alginate salt, it being possible for said aqueous solution to be the continuous aqueous phase of the emulsion according to the invention.

By applying this preparation method the entire amount of polysaccharides is dissolved in the free state, i.e. “non-encapsulated”, in the aqueous phase.

The cosmetically active agent, optionally added, may be outside the liposomes in the aqueous phase of the dispersion of liposomes, or may be partially or completely encapsulated in the liposomes.

In an alternative embodiment, the water of the aqueous suspension of liposomes obtained in step 3 can be eliminated by atomizing or freeze-drying so as to obtain a powder comprising the components of the suspension, said powder then being redispersed in the second aqueous phase comprising at least the non-ionic surfactant.

The liposomess used according to the invention have a diameter of approximately 150 to 250, in particular 150 to 200, Nanometers (nm), as measured by laser granulometry as a suspension of lipid vesicles or by Transmission Electron Microscopy with preparation of the sample by cryofracture.

These liposomes are particularly appropriate for the preparation of cosmetic compositions comprising said liposomes, even in an emulsion, for a cosmetic skin care method for preventing or slowing down the appearance of the effects of skin ageing, in particular of photoageing of the skin, comprising the application, to a part of the skin in need thereof, of a cosmetic composition containing a tocopheryl phosphate as defined above.

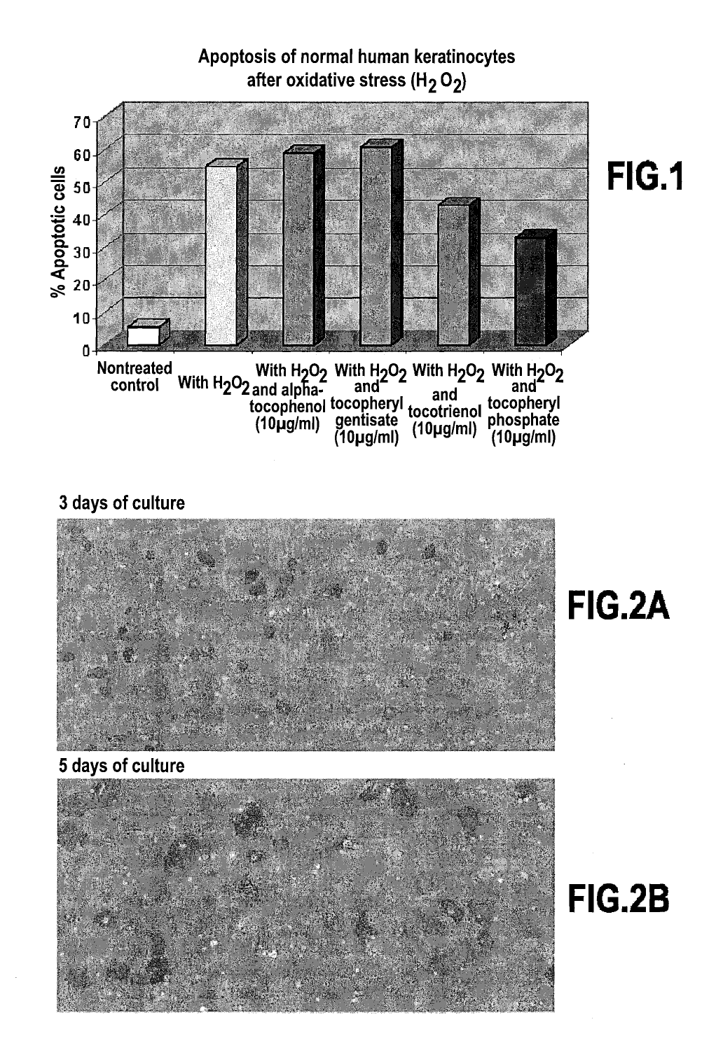

Other characteristics and advantages of the invention appear in the description and the examples which follow and also in FIGS. 1 to 8 to which reference is made in the examples.

FIG. 1, which refers to Example 1, illustrates the effectiveness of alpha-tocopheryl phosphate compared with other vitamin E derivatives as an agent for protecting epidermal stem cells;

FIG. 2 refers to Example 2 and represents the visualisation by videomicroscopy of the keratinocyte colonies derived from the epidermal stem cells at 3 days (FIG. 2A) and 5 days of culture (FIG. 2B);

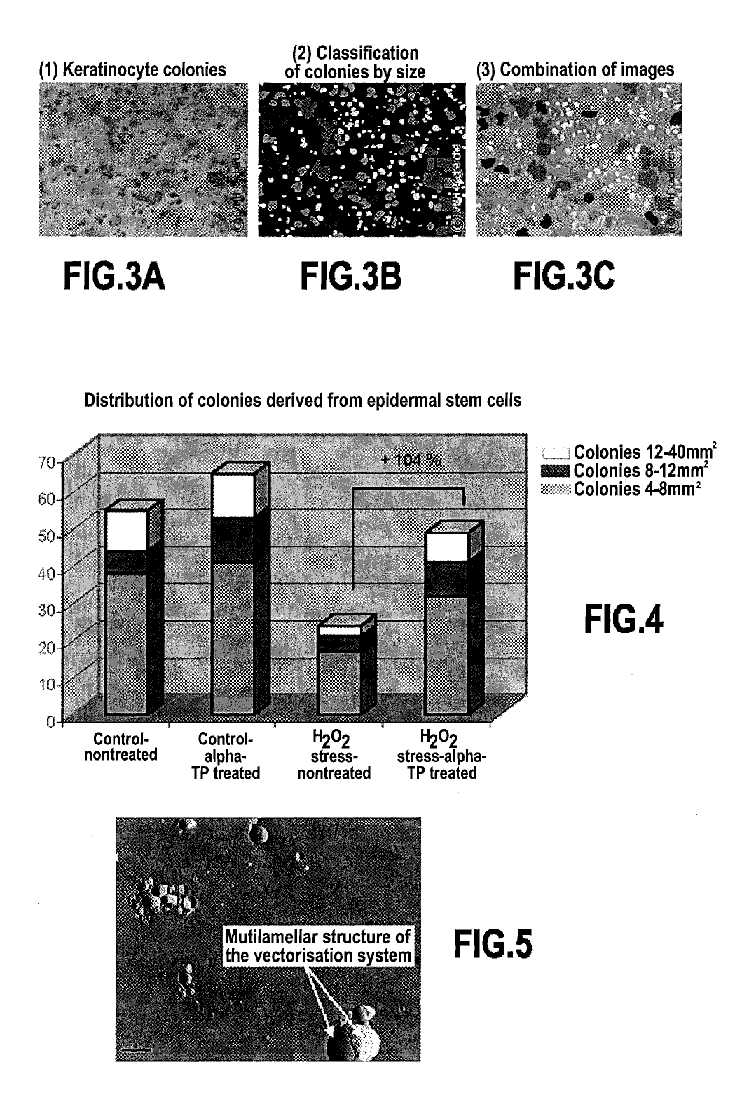

FIG. 3 (FIGS. 3A, 3B and 3C) gives the result of the visualisation of the keratinocyte colonies, FIG. 3A showing these colonies, FIG. 3B giving a classification of the colonies by size, and FIG. 3C, obtained after superimposition of the images, corresponding to FIGS. 3A and 3B;

FIG. 4 refers to Example 2 and gives the distribution of the colonies derived from epidermal stem cells after various treatments;

FIG. 5 is an image obtained by scanning electron microscopy after cryofracture using the serum prepared according to Example 3;

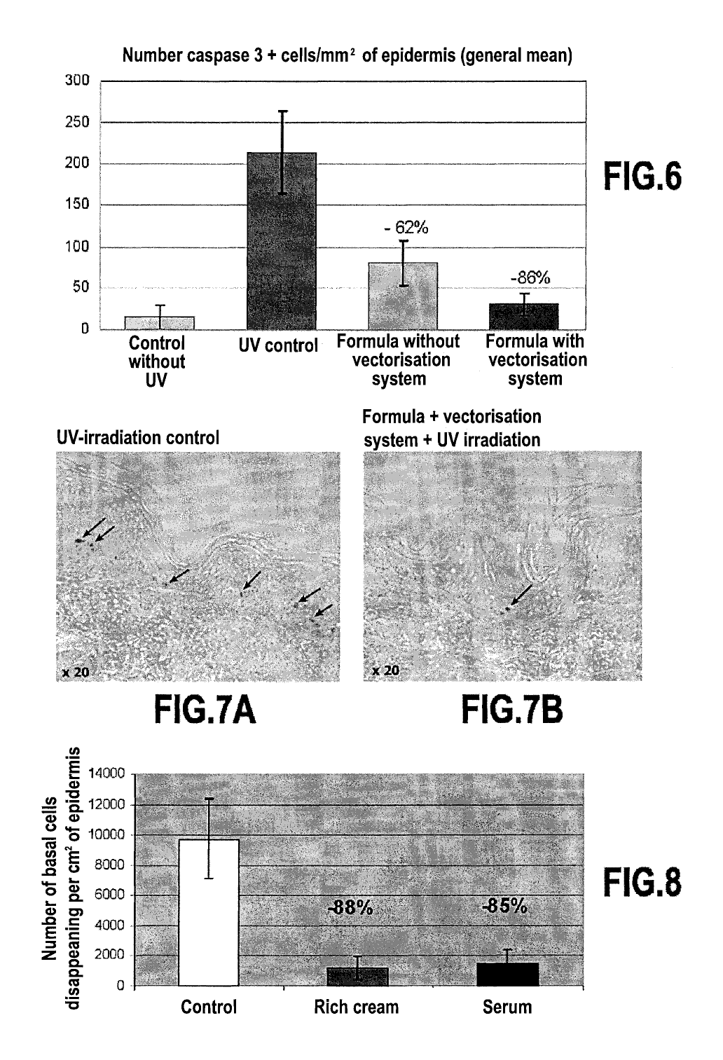

FIG. 6 is given with reference to Example 5 and gives the result of counting caspase 3+ cells after various treatments;

FIG. 7 gives the results of the immunolabelling of caspase 3+ (FIG. 7D) in comparison with a control (FIG. 7A);

FIG. 8 is given with reference to Example 6 and gives the number of basal cells disappearing per cm² of epidermis in the case of a control sample and in the case of two samples treated with alpha-tocopheryl phosphate.

In the examples all percentages are given in weight, the temperature is room temperature, and the pressure is atmospheric pressure, unless otherwise stated.

### EXAMPLE 1

### Comparative Tests Concerning the Protection of Epidermal Stem Cells

The effectiveness of alpha-tocopheryl phosphate relative to other vitamin E derivatives as an agent for protecting epidermal stem cells, located in the basal layer, is compared.

Method

The cells undergoing apoptosis (programmed cell death) are examined on normal human keratinocytes (NHKs) in culture.

The cells are pretreated or not pretreated (controls) for 24 h with the vitamin E derivatives in solution at 10 μg/ml.

The cells are subsequently subjected to oxidative stress (1 mM H₂O₂ for 3 h).

The proportion of cells undergoing apoptosis is determined by cytometry using the fluorescent probes JC-1 (measurement of mitochondrial transmembrane potential) and TOTO-3 (measurement of plasma membrane permeability).

The apoptotic cells are JC-1-negative (corresponding to a drop in the mitochondrial transmembrane potential) and TOTO-3-negative (corresponding to an absence of alteration of the plasma membrane).

This method has been published (Zullani et al. Cytometry part A, 2003, 54A: 100-108).

Results

The measurement results are given in the table below:

| | % NHKs undergoing |
|---|---|
| Experimental conditions | apoptosis |
| Nontreated control | 5.48 |
| Nontreated H₂O₂ control | 54.82 |
| With H₂O₂ and alpha-tocopherol | 58.82 |
| With H₂O₂ and tocopheryl gentisate | 60.67 |
| With H₂O₂ and tocotrienol | 42.85 |
| With H₂O₂ and tocopheryl phosphate | 32.79 |

A lower percentage of cells undergoing apoptosis is noted in the culture of cells treated with the vitamin E phosphate (see FIG. 1 giving the percentage of cells undergoing apoptosis after oxidative stress).

The vitamin E phosphate thus exerts a protective effect against oxidative stress for the keratinocytes, this effect being significantly greater than that observed for the control, and also than that observed for vitamin E or the other derivatives.

### EXAMPLE 2

### Effect of Alpha-Tocopheryl Phosphate on the Protection of Epidermal Stem Cells In Vitro

In order to study these epidermal stem cells in the basal cell layer of the epidermis, the test carried out, known as clonogenecity test, is based on the ability that the stem cells have of adhering, in vitro, to a culture support and of dividing so as to generate a large population of daughter cells grouped together in the form of colonies (Barrandon Y., Green H., Three clonal types of keratinocyte with different capacities for multiplication, J. Cell Biol. 84, 2302-2306 (1987), and Barrandon Y., Biologie des cellules souches épidermiques [Epidermal Stem Cell Biology], Ann. Dermatol. Venereol. Suppl. 2: 285-286, (1998)).

Analysis of the number and of the size of the colonies after culture makes it possible to characterize the epidermal stem cells.

It also makes it possible to evaluate the protective action of an active agent when these cells are undergoing a stress.

The first step of isolating these cells consists in preparing a suspension of epidermal cells (Germain L et al., Improvement of human keratinocyte isolation and culture using thermolysin, Burns 19, 99-104 (1993)).

These cells are subsequently seeded onto feeder cells (3T3 fibroblasts), the mitotic activity of which is blocked with mitomycin.

This living support makes it possible to select the basal cells of the epidermis containing the stem cells and also makes it possible to ensure the growth of the cells with a high dividing capacity, which then form colonies of daughter cells (Barrandon, 1987).

The cells exhibiting the highest dividing capacity form large colonies (>4 mm²).

They correspond to the starting stem cell population.

FIG. 2 corresponds to the visualisation by video microscopy of the keratinocyte colonies derived from the epidermal stem cells at 3 days (FIG. 3A) and 5 days of culture (3B). The colouring of the colonies, herein red, shows the strong growth of certain colonies which correspond to the stem cells.

The protective effect of αTP was demonstrated according to this methodology.

First of all, the keratinocytes are treated with 1 μg/ml of αTP for 24 h, or not treated (controls), and then subjected to oxidative stress (hydrogen peroxide) for 15 minutes.

They are re-seeded at very low density for individualised growth.

After 7 days of culture, the cell colonies formed from each initial keratinocyte are coloured and counted by image analysis according to their size so as to evaluate the amount of stem cells of the initial culture.

FIG. 3 shows a visualisation of the keratinocyte colonies.

The cell colonies are classified by colour according to colony size (blue: greater than 12 mm²; red: from 8 to 12 mm²; orange: from 4 to 8 mm²; yellow: less than 4 mm²). FIG. 3-C represents the superimposition of the images represented in FIG. 3-A (original) and in FIG. 3-B (after colouring). It is thus verified that all the colonies have been taken into account.

The treatment with sodium alpha-tocopheryl phosphate under these conditions makes it possible to significantly maintain a high number of large colonies (>4 mm²), despite the oxidative stress, of +104% compared with the controls where this treatment with alpha-tocopheryl phosphate (alpha-TP) did not take place.

Reference will be made to the graph in FIG. 4 which gives the counts for the various colonies derived from epidermal stem cells as a function of their size: from 4 to 8 mm², from 8 to 12 mm² and greater than 12 mm².

Since the large colonies are derived from stem cells, these results thus indicate that the sodium alpha-tocopheryl phosphate therefore strongly protects this particular keratinocyte population.

### EXAMPLE 3

### Preparation of a Serum Comprising Sodium Alpha-Tocopheryl Phosphate

A serum is prepared according to the following formula (% expressed by weight relative to the total formula):

Phase A

| | |
|---|---|
| Purified water | 60.2% |
| Preservatives | 0.7% |

Phase B

| | |
|---|---|
| Carbomer (Carbopol ® Ultrez 10) | 0.5% |

Phase C

| | |
|---|---|
| Tetrasodium EDTA | 0.2% |
| Sodium hydroxide | 0.2% |
| Glycerol | 3.5% |
| Ascorbic acid | <0.1% |
| 1,3-Butylene glycol | 2.0% |
| Methyl gluceth-20 | 1.8% |
| Purified water | 6.0% |

Phase D

| | |
|---|---|
| Sodium tocopheryl phosphate | 0.2% |
| Sorbitol | 0.3% |
| Sodium alginate | 0.1% |
| Sodium carboxymethylcellulose | <0.1% |
| Polyvinyl alcohol | <0.1% |
| Emulmetik ® 300 IP | 0.5% |
| Emulmetik ® 930 | 0.5% |
| 1,3-Butylene glycol | 1.0% |
| Glycerol | 1.0% |
| Antioxidants | 0.2% |
| Purified water | qs 100% |

The phospholipids of phase D are homogenised with an Ultraturrax.

The phospholipids are subsequently homogenised with the butylene glycol and the glycerol for 20 minutes and the mixture is left to stand for at least 60 minutes.

The other compounds of phase D are added, followed by the purified water.

The lamellar phase obtained is sheared for 20 minutes with an Ultraturrax so as to form a dispersion of liposomes, having a diameter of approximately 150 to 200 Nanometers (nm), as measured by laser granulometry as a suspension of lipid vesicles or by Transmission Electron Microscopy with preparation of the sample by cryofracture.

The sodium alpha-tocopheryl phosphate is included in the serum as an active agent and becomes encapsulated in the liposomes thus prepared.

Separately, the compounds of phase A are heated to 80° C.

At ambient temperature, the compounds of phase B are added, and then the gel is left to swell before adding the compounds of phase C to the gel previously formed.

The dispersion of liposomes is added to the aqueous phase previously prepared.

The composition thus obtained is a serum comprising multilamellar liposomes in which the sodium alpha-tocopheryl phosphate is encapsulated.

As appears in FIG. 5, in the present case, the liposomes comprise several phospholipid layers, which molecules are similar to those of cell membranes.

In this FIG. 5, which is a scanning electron microscopy image obtained on the serum prepared according to the present example, the presence of multilamellar spherical objects corresponding to the vectorisation system is noted.

### EXAMPLE 4

### Rich Cream Comprising Liposomes Encapsulating Sodium Alpha-Tocopheryl Phosphate

Phase A

| | |
|---|---|
| Steareth 2 flakes (Brij ® 72 flakes) | 1.3% |
| Steareth 21 flakes (Brij ® 721P) | 2.2% |
| 95% Cetyl alcohol | 1.2% |
| Stearyl alcohol | 1.2% |
| Stearic acid | 0.35% |
| Palmitic acid | 0.35% |
| Cetyl palmitate | 1.3% |
| Hydrogenated polyisobutene | 5.3% |
| Dicaprylyl carbonate | 4.5% |
| Caprylic/capric triglycerides | 5.0% |
| Dimethicone | 0.2% |
| Cyclopentasiloxane | 2.1% |
| Preservatives | 0.7% |

Phase B

| | |
|---|---|
| Glycerol | 3.5% |
| Purified water | 40.6% |
| Acrylates/C10-C30 alkyl acrylate crosspolymer | 0.5% |

Phase C

| | |
|---|---|
| Tetrasodium EDTA | 0.2% |
| Sodium hydroxide | 0.1% |
| Caprylyl glycol | 0.5% |
| Purified water | 4.8% |

Phase D

| | |
|---|---|
| Sorbitol | 0.4% |
| Sodium alginate | 0.2% |
| Sodium carboxymethylcellulose | <0.1% |
| Polyvinyl alcohol | <0.1% |
| Emulmetik ® 300 IP | 0.5% |
| Emulmetik ® 930 | 0.5% |
| 1,3-Butylene glycol | 1.0% |
| Glycerol | 1.0% |
| Vitamin E phosphate, sodium salt | 0.2% |
| Antioxidants | 0.2% |
| Purified water | qs 100% |

The phospholipids of phase D are homogenised with an Ultraturrax.

The phospholipids are subsequently homogenised with the butylene glycol and the glycerol for 20 minutes and the mixture is left to stand for at least 60 minutes.

The other compounds of phase D are added, followed by the purified water.

The lamellar phase obtained is sheared for 20 minutes with an Ultraturrax so as to form a dispersion of liposomes, having a diameter of approximately 150 to 200 Nanometers (nm), as measured by laser granulometry as a suspension of lipid vesicles or by Transmission Electron Microscopy with preparation of the sample by cryofracture.

Phases A and B are heated to 85° C. separately so as to obtain two homogeneous solutions.

Phase B is subsequently emulsified in oily phase A. The O/W emulsion obtained is gradually cooled with stirring, and then, at 70° C., the compounds of phase C are added, in particular to neutralise the polymers.

The dispersion of liposomes is added to the O/W emulsion previously prepared, with stirring and without incorporating air.

The emulsion obtained comprises multilayer liposomes in the continuous aqueous phase.

These liposomes are not destroyed by the action of the surfactants stabilising the emulsion.

### EXAMPLE 5

### Effect of Alpha-Tocopheryl Phosphate (αTP) Encapsulated in Liposomes on the Protection of Epidermal Basal Cells on a Skin Explant, after UV Attack

The advantage of the encapsulation of the sodium alpha-tocopheryl phosphate in liposomes is compared here.

The serum comprising the sodium alpha-tocopheryl phosphate is prepared in accordance with Example 3.

For the test, a serum devoid of liposomes (phase D of the previous example), in which the sodium alpha-tocopheryl phosphate is solubilised in phase B, is prepared.

Method

Tissue from plastic surgery on a 36-year-old woman (Caucasian, P495AB36) was cut up into 15 explants 10 mm in diameter. These explants are kept alive in BEM medium (BIO-EC's Explants Medium).

1. Treatment

At time 0, the explants are placed in the survival medium (2 ml/explant) and the treatment is applied:
The explants are treated twice a day, by topical application (2 mg/explant) for 2 days before irradiation.
On the day of irradiation, one application is carried out 3 h before the irradiation and one application just after irradiation.
The following day, an application is carried out 3 h before the end of the survival period.
The controls do not receive any treatment.

2. Irradiation

The irradiation system is an Oriel solar simulator, normally used to determine the sun protection factor (SPF) and for photosensitisation studies.

At day 2, before irradiation, the survival medium is replaced with HBSS buffer (Hank's Balanced Saline Solution). The irradiation dose is 7 J/cnn².

Just after the irradiation, the explants are again placed in the survival medium. The nonirradiated explants are placed in the dark during the irradiation and their medium is also replaced with HBSS buffer.

3. Samples

At day 3, 24 h after the end of irradiation, the explants are removed and fixed in formol buffer.

4. Immunolabelling of Active Caspase-3

The explants are dehydrated in a Leica automat TP 1020, embedded in paraffin (Leica EG 1160 embedding station automat).

Sections 5 μm thick are cut on a microtome (Leica Minot type microtome RM 2125) and placed on silanised slides.

The caspase-3 immunolabelling of the cells is carried out on the sections using a polyclonal anti-active caspase-3 antibody (rabbit, Chemicon ref AB3623) which recognises the active form of caspase-3.

The labelling is revealed using a Vectastain universal ABC VECTOR amplifier system kit with DAB staining.

The images are taken on a Nikon TE2000 videomicroscope under transmitted light for the immunolabelling combined with a phase-contrast image for measuring the surface of the epidermis. The caspase-3-positive cells are counted by image analysis on the LEICA QWIN software and quantified relative to the surface of the epidermis.

Results

The caspase-3+ cells are counted on the samples of cells treated with the formulations tested.

The results are represented in FIG. 6, which gives the number of epidermal basal cells disappearing in fragments of skin exposed to a complete solar spectrum. These cells are identified by immunohistochemistry using an antibody which recognises active caspase-3, a marker for apoptotic cells.

FIG. 7 gives the result of the immunolabelling of active caspase-3 on skin explants exposed to a complete solar spectrum. Only the basal cells of the epidermis are labelled. A smaller number of positive cells is noted in the epidermis treated with the vectorisation system.

We demonstrated that a composition containing the vectorisation system makes it possible, when it is applied to human skin samples kept alive ex vivo and exposed to a complete solar spectrum, to significantly increase the protective efficacy of sodium alpha-tocopheryl phosphate with respect to the epidermal stem cells located in the basal layer (FIGS. 6 and 7).

### EXAMPLE 6

### Effect of alpha-tocopheryl phosphate (αTP=TP vityl) encapsulated in liposomes on the protection of epidermal basal cells on a skin explant, after UV attack

The protective effect, with respect to epidermal stem cells, of cosmetic compositions comprising the alpha-tocopheryl phosphate encapsulated in liposomes and prepared according to Examples 3 and 4 of the present patent is evaluated.

These compositions are a serum and an oil-in-water emulsion in which the continuous aqueous phase comprises said liposomes. The same test as in Example 5 is carried out on these compositions.

The results are reproduced in FIG. 8, which represents the number of basal keratinocytes of the epidermis which disappear during acute exposure with a complete solar spectrum after treatment with the serum or the rich cream comprising the tocopheryl phosphate encapsulated in liposomes. These cells are identified by indirect immunofluorescence using anti-active caspase-3 antibody, active caspase-3 being a marker for apoptotic cells.

We demonstrated that the rich cream and the serum containing 0.2% of αTP, applied to fragments of skin, reduce by more than 80% the number of basal cells of the epidermis which disappear after acute exposure with a complete solar spectrum.

1. A method of skin care for slowing down the appearance of the effects of skin ageing, comprising applying to areas of skin in need thereof, a cosmetic composition comprising a tocopheryl phosphate component selected from tocopheryl phosphate, a cosmetically acceptable tocopheryl phosphate salt and a cosmetically acceptable tocopheryl phosphate ester, said tocopheryl phosphate being at least partially encapsulated in liposomes comprising a phospholipid membrane comprising a mixture of two different fractions of soybean lecithin, a first lipid fraction of soybean lecithin comprising a mixture of phospholipids comprising more than 90% of phosphatidyl choline, and a second lipid fraction comprising a mixture of phospholipids comprising between 15% and 30% by weight of phosphatidylcholine.

2. The method of claim 1, wherein the composition of the second fraction comprises, in weight %:
From 10 to 20% of Phosphatidyl ethanolamine
From 10 to 15% of Phosphatidyl inositol
From 15 to 30% of Phosphatidyl choline
From 5 to 10% of phosphatidic acid
Less than 1% of Lyso Phosphatidyl choline.

3. The method of claim 1, wherein the composition of the second fraction comprises, in weight %:
From 13 to 18% of Phosphatidyl ethanolamine
From 12 to 15% of Phosphatidyl inositol
From 22 to 30% of Phosphatidyl choline
From 5 to 10% of phosphatidic acid
Less than 1% of Lyso Phosphatidyl choline.

4. The method of claim 1, wherein said liposomes are multilamellar liposomes.

5. The method of claim 1, wherein said liposomes have an average size of 150 to 250 nanometers (nm) as measured by laser granulometry as a suspension of liposomes or by Transmission Electron Microscopy with preparation of the sample by cryofracture.

6. The method of claim 1, wherein the liposomes further comprise amphiphilic lipids selected from the group consisting of phospholipids, phosphoaminolipids, glycolipids and mixtures thereof.

7. The method of claim 1, wherein the liposomes further comprise phospholipids selected from the group consisting of phosphatidylserine, phosphatidylethanolamine, phosphatidylglycerol, phosphatidylinositol, and any mixtures thereof.

8. The method of claim 1, wherein the liposomes further comprise amphiphilic lipids chosen from the group consisting of sphingomyelin, cerebrosides and oxyethylenated polyglyceryl stearates.

9. The method of claim 1, wherein said composition further comprises at least one first polysaccharide in water-soluble form.

10. The method of claim 1, wherein the composition is in the form of an emulsion comprising a continuous aqueous phase and a dispersed fatty phase, wherein said aqueous phase contains said liposomes in the presence of a non-ionic surfactant selected from a fatty alcohol polyethylene glycol ether and a fatty alcohol polypropylene glycol ether, and any mixture thereof, and of at least one first polysaccharide in water-soluble form.

11. The method of claim 10, wherein said non-ionic surfactant is selected from a fatty alcohol polyethylene glycol ether and any mixture thereof.

12. The method of claim 10, wherein the non-ionic surfactant comprises at least one stearyl alcohol polyethylene glycol ether.

13. The method of claim 10, wherein said non-ionic surfactant is a mixture of ethoxylated compounds of stearyl alcohol of formula (A):
CH3(CH2)17(OCH2CH2)nOH (A).

14. The method of claim 9, wherein said first polysaccharide in water-soluble form is selected from the group consisting of a starch, a cellulose, a pectin, a gum, an alginate, a dextran, a carragheenate and hyaluronic acid.

15. The method of claim 9, wherein said first polysaccharide in water-soluble form is an alkali metal alginate.

16. The method of claim 9, wherein said composition comprises said first polysaccharide comprising at least one alginate in water-soluble form, and a second polysaccharide in water-soluble form different from said first polysaccharide.

17. The method of claim 16, wherein the second polysaccharide is an alkali metal salt of carboxymethylcellulose.

18. The method of claim 16, wherein the total amount of polysaccharide in water-soluble form is effective for protecting the liposomes against their degradation under the effect of at least one surfactant.

19. The method of claim 16, wherein the total amount of water-soluble polysaccharide is between 0.1% and 10% by weight of the composition.

20. The method of claim 9, wherein the [phospholipids/water-soluble polysaccharide] ratio of the composition is between 0.1 and 20.

21. The method of claim 9, further comprising at least one hydrophilic polymer other than said water soluble polysaccharide.

22. The method of claim 21, wherein said hydrophilic polymer is selected from the group consisting of polyvinylpyrrolidone and polyvinyl alcohol, and any mixture thereof.

23. The method of claim 1, further comprising at least one water-soluble hydrophilic compound selected from the group consisting of C₆ or C₁₂ sugars, C₆ or C₁₂ sugar ethers, C₆ or C₁₂ sugar esters and mixtures thereof.

24. The method of claim 10, wherein the fatty phase of said emulsion contains at least one triglyceride.

25. The method of claim 24, wherein the fatty phase of said emulsion is a continuous phase for further inclusion of a dispersed water phase, thereby forming itself a water-in-oil (W/O) emulsion.

26. The method of claim 10, further comprising at least one cosmetically active agent contained at least in part either in the aqueous phase, or in the fatty phase of the emulsion, or in both phases, and at least one cosmetically acceptable excipient.

27. The method of claim 1, wherein said tocopheryl phosphate component is alpha-tocopheryl phosphate.

28. The method of claim 1, wherein said tocopheryl phosphate is chosen from the group consisting of the alkali metal salts, the alkaline earth metal salts, and the ammonium salts or salts of primary, secondary or tertiary amines.

29. The method of claim 1, which is a skin care selected from slowing down the formation of wrinkles on the face, and reducing or smoothing out the wrinkles already formed.
